(19) United States
(12) Patent Application Publication
(10) Pub. No.: US 2008/0058717 A1
(43) Pub. Date: Mar. 6, 2008

(54) ORAL CAVITY LIQUID DELIVERY SYSTEM INCLUDING PRE-ANGLED NEEDLE GUIDANCE ASSEMBLY AND METHOD FOR USING THE SAME

(76) Inventor: David M. Spector, Roseville, CA (US)

Correspondence Address: CRAIG A. SIMMERMON, 251 LYMAN CIR., SACRAMENTO, CA, 95835, US

(21) Appl. No.: 11/309,646

(22) Filed: Sep. 4, 2006

(51) Int. Cl. A61M 5/00; A61M 5/32

(52) U.S. Cl. 604/117; 604/272

(57) ABSTRACT

A delivery system for dispensing sterile liquids into the oral cavity using a novel pre-angled needle guidance assembly attached to a conventional anesthetic needle and conventional anesthetic syringe loaded with a novel cartridge ampule containing a certain liquid solution. This system substantially improves the efficiency of delivering small amounts of sterile fluid to the oral cavity by hypodermic means or by irrigation means using pre-angled needle guidance assemblies to temporarily bend needles or cannulas in order to reach otherwise impossible-to-reach locations within the oral cavity while also providing the ability to dispose of used needles without re-bending and thus complying with federal OSHA, state OSHA, and state regulatory disposal regulations.

### BACKGROUND OF INVENTION

Devices currently available to dental practitioners used to deliver small amounts, less than two cubic centimeters, of sterile irrigation liquid or anesthetic solution into the oral cavity are prone to problems related to needle disposal safety; effective, efficient, and ergonomic use by the practitioner; high cost; timely set-up requirements; and other issues. This invention combines a novel needle guidance sleeve assembly, a novel cartridge ampule, and a conventional anesthetic syringe or standard intraligamental syringe to yield an oral cavity liquid delivery system that is surprisingly simple, extremely effective, easy to use, inexpensive, and capable of delivering liquid to oral cavity locations that are currently impossible to do so without resorting to illegal needle bending and disposal methods. Specifically, this invention utilizes a “pre-bent” rigid needle guidance sleeve in conjunction with a cartridge ampule containing sterile fluid which allows the practitioner to efficiently, effectively, and legally complete many procedures such as intrapulpal injection (depicted in FIG. 11), intraligamental injection (PDL) technique (depicted in FIG. 12), anesthetizing accessory nerves on the lingual aspect of the lower posterior teeth, various endodontic sterile irrigation procedures, and sterile irrigation procedures conducted during various oral surgical procedures, including, but not limited to, tooth extraction, periodontal surgery, endodontic surgery, biopsy procedures, implant surgery, and restorative dental procedures.

With anesthetic delivery, certain procedures physically require the use of angled needles to reach certain locations in the oral cavity. As there are no pre-bent needles available to the practitioner through current supply channels, the practitioner must bend a straight needle himself. In this instance, the practitioner or his employee must then re-bend the used needle in order to re-cap the needle before disposal or, even worse, simply dispose the needle in a bent and uncapped condition. However, federal and state OSHA regulations commonly call for needles to be capped before disposal and for needles not to be bent or broken for the purpose of disposal. Therefore, whether the practitioner re-bends to re-cap or disposes without re-capping, the practitioner is not in compliance with federal and state OSHA regulations when he bends a needle to deliver anesthetic or for any other purpose.

The current invention includes a pre-bent rigid needle guidance assembly, in a variety of angles, through which a standard needle is inserted. The practitioner simply slides the appropriate pre-angled needle guidance assembly onto the distal end of a standard needle hub assembly, completes the anesthetic delivery procedure, removes the guidance sleeve, re-caps the needle assembly, and disposes the needle. As with standard straight needle disposal, disposal of needles used with the current invention does not require re-bending in order to re-cap, as the standard needle rebounds to a nearly straight condition after sleeve removal, finally allowing practitioners to dispose of needles legally and properly.

A major benefit of the current invention is the substantial reduction of the risk of puncture wounds to the dentist and his staff. Needle puncture wounds create a risk of spreading infectious disease and also require the dentist to initiate a very complex and costly regulatory procedure designed to limit the risks created by such accidental needle stick incidents. The current invention would substantially reduce the risk of occurrences of such incidents.

The most common small-volume oral cavity irrigation procedure used by dental practitioners is the manual method. Whether the practitioner starts with straight or pre-bent cannulas, this irrigation procedure is prone to problems because of complex and costly set-up requirements. Set-up consists of assembling a large bottle of sterile liquid, a sterile scissor to open the hermetically sealed bottle, a sterile plastic syringe, a sterile cannula, and a sterile vessel in which to pour the sterile liquid so that it may be extracted into the sterile syringe. Manual irrigation methods involve pouring solutions out of a large bottle into a small cup or container placed on a countertop or bracket table from which the practitioner fills and re-fills the syringe. This procedure is unnecessarily time consuming and costly as compared to the procedure used with the current invention.

Additionally, many irrigants are caustic, as with sodium hypochlorite, chloroform, EDTA, hydrogen peroxide, and ethyl alcohol, which can be spilled or splashed onto the patient, dentist, staff, work surface, or equipment which can cause substantial human injury, costly equipment damage, and/or costly clean-up procedures. The proposed invention would eliminate the occurrence of all of these circumstances.

Another common sterile irrigation system is a countertop bag and tube apparatus, such as model NWS-9 sold by Aseptico. This type of system is even more costly and more cumbersome than the manual system. Hence, the current invention is an improvement over this method as well.

Unfortunately, another very common method of delivering irrigants to the oral cavity actually introduces “un-sterile” irrigants into the patent's system. Many practitioners are using the air/water syringe attached to the dental chair unit to perform irrigation procedures. Typically, practitioners maintain and clean the internal plumbing, i.e. water reservoir, tubing, and hand piece, of the dental unit by flushing these items with water at the beginning of each day and in-between patients. Practitioners do not autoclave dental unit components which come in contact with irrigation solutions. This is unfortunate because dental units are known to become contaminated with “biofilm” and live bacteria after only short periods of use and such contaminants are not eliminated by standard water flushing. Therefore, practitioners are commonly creating risks of infection during irrigation procedures by using non-sterile irrigation water from their dental units.

Pre-bent irrigation cannulas are available to the practitioner according U.S. Pat. No. 6,079,979 (Riitano); however, Riitano differs substantially from and is inferior to the proposed invention. Firstly, Riitano is only attachable to conventional syringes of a Luer Lock design. Luer Lock syringes are cumbersome to use as compared to the anesthetic syringe which provides substantially better ergonomics, yielding much more effective control and ease of use, because of its heavier weight and “feel” for which dentists generally have become accustomed. This invention includes a conventional anesthetic syringe while Riitano cannot be attached to this type of syringe. Secondly, Riitano cannot accept cartridge ampules. One of the significant problems in the field of dentistry that this invention solves is the inability of current systems to allow for quick, inexpensive, efficient, and easy delivery of small volumes of sterile irrigation liquid into the oral cavity. The plunger type tubular cartridge element of this invention is the primary facilitating element of this feature; while Riitano cannot accept any type of cartridge ampule. This is another significant distinction. Specifically, Riitano cannot accept cartridge ampules because it lacks a proximal end needle as well as other limitations. Additionally, Riitano is used only for irrigation and cannot be used where a sharp penetrating needle is required to deliver an injection.

### BRIEF DESCRIPTION OF THE DRAWINGS

Typical embodiments of the present invention are illustrated in the accompanying drawings which are not to be limiting of the invention's scope in which:

FIG. 1 is a side view of pre-angled needle guidance assembly with outer layer and is the preferred embodiment of pre-angled needle guidance assembly.

FIG. 2 is a side view of pre-angled needle guidance assembly without outer layer.

FIG. 3 is a longitudinal section of an anesthetic needle “docked” into pre-angled needle guidance assembly and is the embodiment used for blunt-tipped irrigation procedures.

FIG. 4 is a is a longitudinal section of an anesthetic needle “docked” into pre-angled needle guidance assembly and is the embodiment used for sharp-tipped injection procedures. This is the preferred embodiment of pre-angled needle guidance assembly assembly.

FIG. 5 is a side view of oral cavity liquid delivery system including pre-angled needle guidance assembly.

FIG. 6 is a perspective view of needle docking station depicted with pre-angled needle guidance assembly mounted into the keyhole mounting slot in preparation for docking of an anesthetic needle.

FIG. 7 is a plan view of needle docking station.

FIG. 8 is a perspective view of cartridge ampule with faceted distinguish ability means.

FIG. 9 is a bottom end view of cartridge ampule with faceted distinguish ability means.

FIG. 10 is a top end view of cartridge ampule with faceted distinguish ability means.

FIG. 11 is a depiction of an intrapulpal injection procedure or irrigation procedure using the preferred embodiment of oral cavity liquid delivery system including pre-angled needle guidance assembly.

FIG. 12 is a depiction of an intraligamental injection procedure using the preferred embodiment of oral cavity liquid delivery system including pre-angled needle guidance assembly.

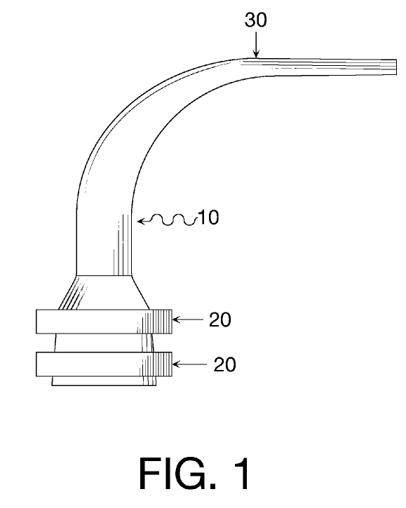

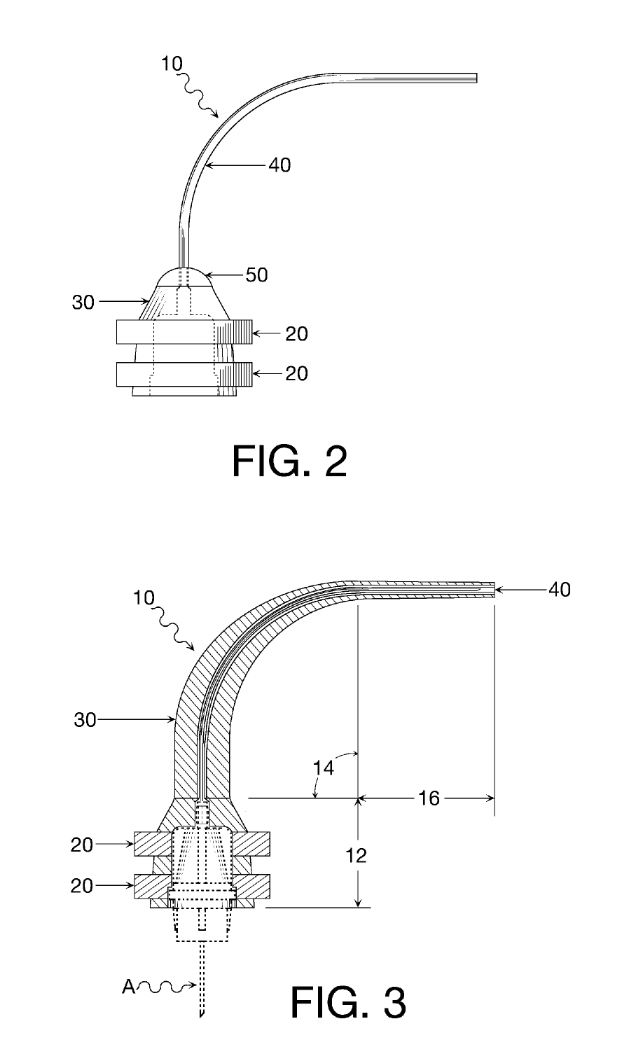

### DEFINITION LIST 1

| Term | Definition |
|---|---|
| 10 | Pre-angled needle guidance assembly |
| 12 | Proximal portion of pre-angled needle guidance assembly |
| 14 | Angled portion of pre-angled needle guidance assembly |
| 16 | Distal portion of pre-angled needle guidance assembly |
| 20 | Retention ridges used to mount assembly onto needle docking |
| | station |
| 30 | Rigid material layer |
| 40 | Needle guidance tube |
| 50 | Adhesive |
| 60 | Needle docking station |
| 70 | Conventional anesthetic syringe assembly |
| 80 | Distal needle safety cap of conventional aesthetic needle assembly |
| 90 | Needle safety cap port hole |
| 100 | Keyhole mounting slot |
| 110 | Cartridge ampule with faceted distinguishability means |
| 120 | Collar of cartridge ampule |
| 130 | Flexible membrane of cartridge ampule |
| 140 | Piston of cartridge ampule |
| 150 | Tubular body of cartridge ampule |
| A | Conventional anesthetic needle assembly |

### DETAILED DESCRIPTION OF INVENTION

The oral cavity liquid delivery system, depicted in FIG. 5, utilizes conventional anesthetic syringes 70 or conventional intraligamental syringes, novel cartridge ampules 110 filled with anesthetic or irrigation solution that are attachable to the syringe 70, conventional needle hub assemblies A also attachable to the syringe, novel pre-angled needle guidance assemblies 10 attachable to the needle hub assembly A as well as the needle docking station 60, and a novel needle docking station 60 used to assist with the attachment and removal of the pre-angled needle guidance assembly 10 to the needle hub assembly A, as well as the removal and attachment of a distal needle safety cap 80 to a conventional anesthetic needle hub assembly A.

The practitioner chooses from the system a cartridge ampule 110 containing the appropriate liquid for the procedure at hand and inserts the ampule 110 into the syringe 70. The system allows for ampules containing various anesthetics and irrigation solutions.

Novel cartridge ampules 110 of the current invention contain solution other than anesthetic, phosphoric acid in certain concentrations, or hydrocolloid impression material. These cartridges will have a means to distinguish them (see claim 11) from other types of cartridges used for injection purposes already in the public domain. First is the faceted means where the cartridge has a smooth round inner bore, as with currently available cartridges, and a faceted exterior surface, i.e. hexagon, octagon, decagon, or other polygonal shape, with overall diameter equal to that of a standard cartridge to permit fitting the cartridge into a syringe. The faceted shape will alert the operator to the fact that the cartridge ampule does not contain anesthetic. Second is the decal means where the cartridge ampules are covered with an opaque boldly colored decal that makes the cartridges pertaining to this invention easily distinguishable from anesthetic cartridges. The label decal will have one or more longitudinal narrow slits that are transparent to allow the operator to determine how much liquid remains within the carpule. Third is the ink means where the cartridge includes areas applied with ink or paint that may contain bar codes and will contain signification that cartridge ampules with ink means are not intended for injection purposes. There is also a combination means where the cartridges include the faceted means, decal means and/or ink means to prevent confusion between novel cartridges pertaining to this invention and non-anesthetic cartridges already in the public domain. Other equivalent distinguish ability means are also included with this invention. The best mode is the combination means.

After loading the appropriate cartridge ampule 110 into the syringe 70, the practitioner chooses from the system a pre-angled needle guidance assembly 10 with the appropriate angle and tube 40 length to perform the procedure at hand and attaches the pre-angled needle guidance assembly 10 to the needle assembly A. The practitioner then “mounts” or slides the pre-angled needle guidance assembly 10 onto the anesthetic needle A. This mounting process is accomplished through the use of a needle docking station 60. The preferred embodiment includes an angle of ninety degrees.

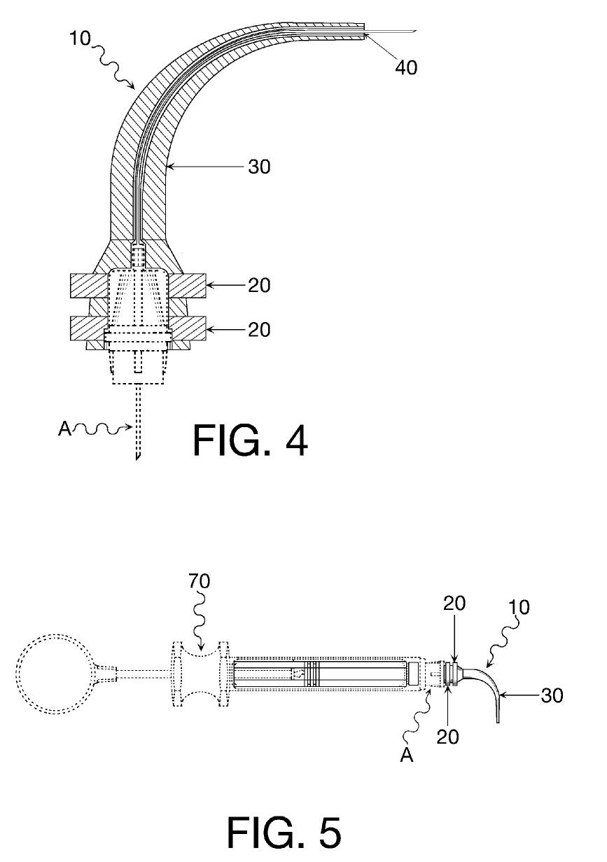

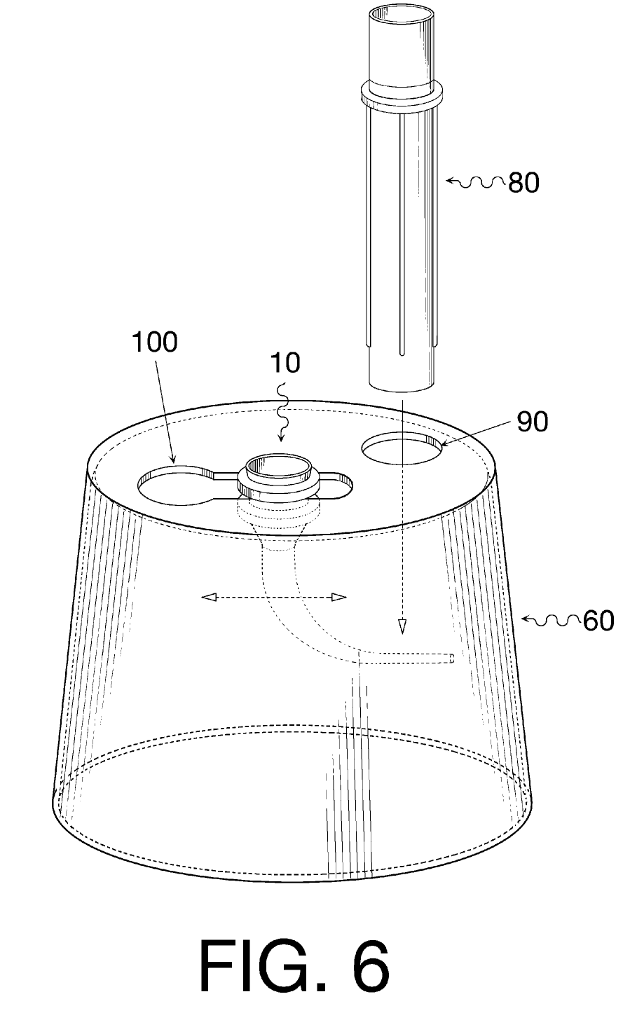

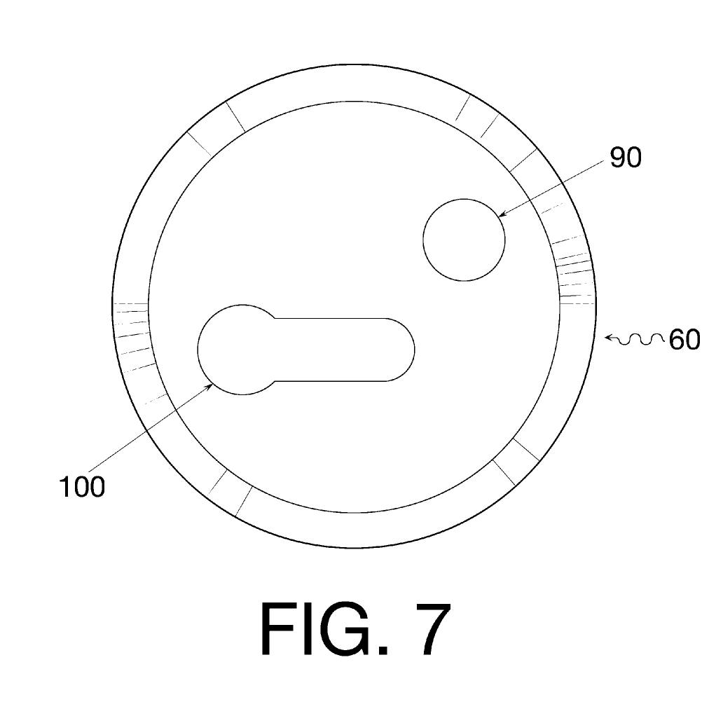

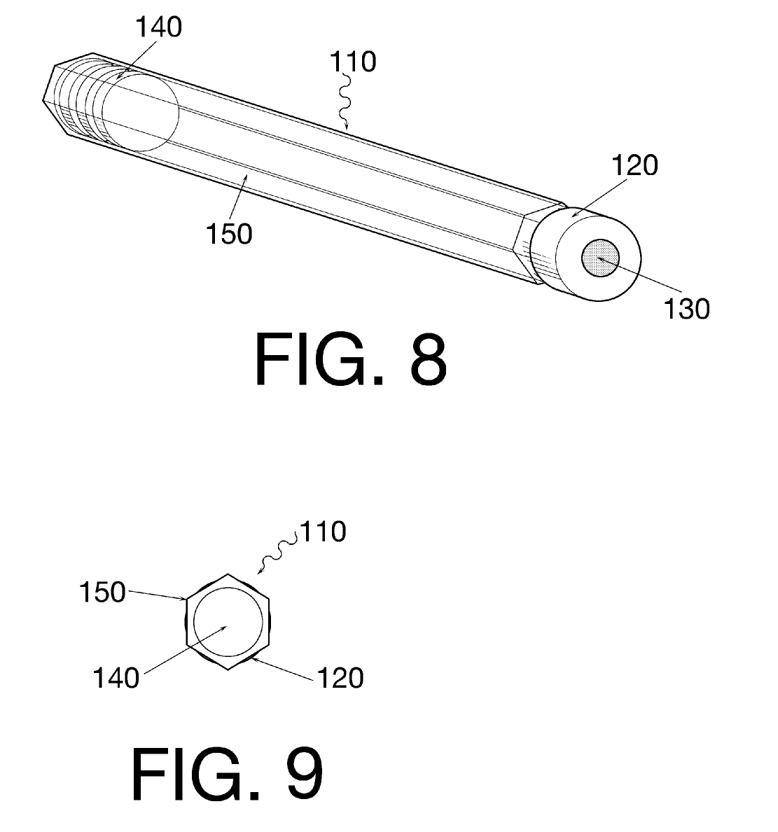

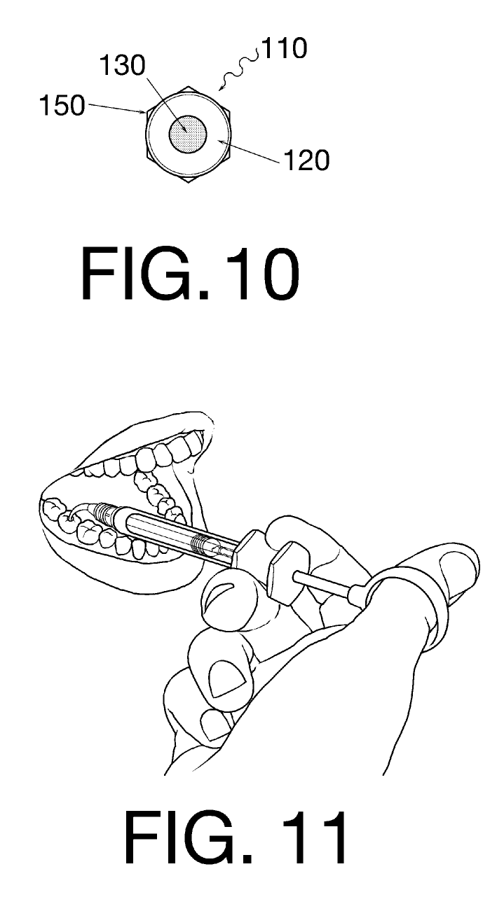

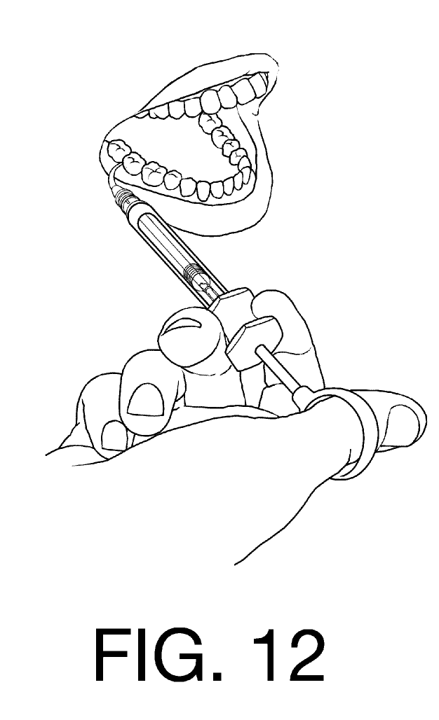

The distal ends of conventional anesthetic needles A come in two lengths: 25 millimeters or “short” and 35 millimeters or “long”. Pre-angled needle guidance assemblies 10 shall also be manufactured in different lengths. More specifically, the invention shall include different tube 40 lengths to accommodate either short or long conventional needles, or any other length needles that are currently sold. The invention shall include at least four standard tube 40 lengths: two each for both short and long conventional needle configuration. The tube length of pre-angled needle guidance assemblies intended for irrigation procedures, depicted in FIG. 3, shall exceed the length of the anesthetic needle, either short or long, by essentially one millimeter. This will have the effect of converting a sharp piercing injection needle into a blunt angled irrigating tip. The tube length of pre-angled needle guidance assemblies intended for injection procedures, depicted in FIG. 4, shall essentially be 5-10 millimeters shorter than the conventional needle, either short or long, thereby leaving the piercing needle tip exposed. This configuration is the best mode because it allows the practitioner to reach hard-to-access areas in the oral cavity with the use of an “angled” needle and also permits the practitioner to deliver intraligamental injections (depicted in FIG. 12), intrapulpal injections (depicted in FIG. 11), and carry out irrigation procedures in repeated succession without changing instruments. The best mode length of the tube is 26 millimeters and the best mode radius of the tube is 12 mm, which is designed to mount on a conventional long anesthetic needle assembly.

The needle attachment means (see claim 2) may consist of: a Monoject means wherein the concave surface at the proximal end of the proximal portion of the pre-angled needle guidance assembly is shaped to cooperate accurately with the convex shape of the distal side of any conventional anesthetic needle hub distributed by Monoject, thereby creating a “slip fit” at the land area where the pre-angled needle guidance assembly reversibly attaches to the needle assembly; a Sepdodont means wherein the concave surface at the proximal end of the proximal portion of the pre-angled needle guidance assembly is shaped to cooperate accurately with the convex shape of the distal side of any conventional anesthetic needle hub distributed by Sepdodont, thereby creating a “slip fit” at the land area where the pre-angled needle guidance assembly reversibly attaches to the needle assembly; or any other functionally equivalent needle attachment means including but not limited to the needle attachment means to attach all other anesthetic needles currently manufactured. Best mode is the Monoject means.

During mounting of the pre-angled needle guidance assembly, the straight anesthetic needle A is guided around the radius of tube 40 of the pre-angled needle guidance assembly 10. This action creates a tendency of the needle to resist deformation which creates friction between the external surface of the needle A and the lumen of the of the tube 40 thereby creating retention and stability. These sources of resistance and retention provide sufficient stability of the pre-angled needle guidance assembly 10 to carry out irrigation procedures, intraligamental injections, and intrapulpal injections. Also note these sources of resistance are also present when removing the needle from the pre-angled needle guidance assembly.

The tube attachment means (see claim 2) shall consist of: an adhesive means where the tube 40 is permanently secured to the concave surface at the proximal end of the proximal portion of the pre-angled needle guidance assembly 12 by adhesive 50; a molded means where the tube 40 is permanently secured to the concave surface at the proximal end of the proximal portion of the pre-angled needle guidance assembly 12 by molding or casting the rigid material layer 30 around the tube; or other equivalent means. With all tube attachment means, the lumen of the tube “funnels” into the concave surface area of the hub so that the merger between concave surface area and lumen is smooth and contiguous. The molded means is the preferred embodiment.

After the pre-angled needle guidance assembly 10 is attached to the needle assembly A, the practitioner performs the specific oral cavity liquid delivery procedure or procedures, removes the pre-angled needle guidance assembly, that is done through the use of the needle docking station 60, and then re-caps the needle using the single-handed “scoop” technique or a needle re-capping device, such as the needle docking station 60. With the current invention, the needle assembly is disposed of without directly touching the needle itself. Therefore, the needle is disposed of legally and safely. Additionally, the risk of needle puncture is greatly reduced.

In the case of irrigation procedures, the practitioner can quickly and easily change irrigation solutions by simply changing cartridge ampules. There is no complex and costly set-up as with the manual delivery or bag-and-tube systems. There is no risk of spilling. Most importantly, there is no risk of contaminating the patent's oral cavity with non-sterile irrigants.

What is claimed is:

1. A pre-angled needle guidance assembly, comprising: a tube having a lumen running therethrough; a distal portion; a proximal portion; and an angled portion; wherein said tube is one continuous member throughout said distal and angled portions and said angled portion of pre-angled needle guidance assembly joins said distal portion of pre-angled needle guidance assembly to said proximal portion of pre-angled needle guidance assembly at an angle.

2. A pre-angled needle guidance assembly as in claim 2, wherein said proximal portion of pre-angled needle guidance assembly comprises: a concave surface at the proximal end; a tube attachment means to connect said concave surface to said tube so that said concave surface is essentially seamlessly connected to said lumen in said tube; and a needle attachment means to reversibly connect said proximal portion of pre-angled needle guidance assembly to an anesthetic needle assembly.

3. A pre-angled needle guidance assembly as in claim 3, wherein said proximal portion of pre-angled needle assembly further comprises: two ridges protruding radially outward, sized and spaced to cooperate accurately with the keyhole mounting slot on the needle docking station, thereby allowing said ridges freely pass through the wide opening of the keyhole mounting slot and create a “slip fit” between said ridges and both the narrow opening of the keyhole mounting slot and the thickness of said horizontal member.

4. A pre-angled needle guidance assembly as in claim 3, wherein the length of said tube ranges from 10-50 millimeters.

5. A pre-angled needle guidance assembly as in claim 4, wherein said tube in angled portion of pre-angled needle guidance assembly has radius ranging from 3-40 millimeters and said angle ranges from 5-175 degrees.

6. A pre-angled needle guidance assembly as in claim 5, wherein said tube consists of metal, plastic, ceramic, fiberglass, or any combination thereof.

7. A pre-angled needle guidance assembly as in claim 6, wherein said proximal portion of pre-angled needle guidance assembly consists of metal, plastic, ceramic, fiberglass, or any combination thereof.

8. A pre-angled needle guidance assembly as in claim 7, wherein said angled portion of pre-angled needle guidance assembly and said distal portion of pre-angled needle guidance assembly further comprise an outer layer made of rigid material fully encapsulating said tube.

9. A pre-angled needle guidance assembly as in claim 8, wherein said outer layer consists of metal, plastic, ceramic, fiberglass, or any combination thereof.

10. A plunger-type tubular cartridge ampule containing certain sterile fluid other than: 1) anesthetic, 2) phosphoric acid in 37% solution, 3) phosphoric acid in 50% solution, or 4) hydrocolloid impression material.

11. A plunger-type cartridge ampule as in claim 10, further comprising: a tubular body; a piston, wherein said piston is movable axially across the long axis of said tubular body and seals and contains said certain sterile fluid within said tubular body; a cap, wherein said cap further comprises a collar and flexible membrane both secured to said tubular body and seals and contains said certain sterile fluid within said tubular body; and a means to distinguish said cartridge ampule from other cartridge ampules already in the public domain containing anesthetic or other fluid intended for injection purposes.

12. A plunger-type cartridge ampule as in claim 11, wherein said certain sterile fluid is any one of the group consisting of sterile water, sterile saline solution, sodium hypochlorite, chloroform, EDTA solution, hydrogen peroxide, ethyl alcohol, eucalyptol, and chlorhexidine gluconate.

13. A device to support said pre-angled needle guidance assemble, comprising: a vertical support member; and a horizontal support member connected along its perimeter to said vertical support member, comprising: a flat layer of rigid material defining a keyhole mounting slot therethrough used to reversibly attach said pre-angled needle guidance assembly and further defining a needle safety cap port hole used to reversibly attach an anesthetic needle assembly distal safety cap.

14. A device to support said pre-angled needle guidance assembly as in claim 13, wherein said flat layer further defines said keyhole mounting slot to cooperate accurately with said ridges on said proximal portion of pre-angled needle guidance assembly, thereby allowing said ridges to freely pass through the wide opening of said keyhole mounting slot and reversibly mount onto the narrow opening of said mounting slot, yielding a “slip fit” between said ridges and both the narrow opening of said keyhole mounting slot and the thickness of said horizontal member.

15. A device to support said pre-angled needle guidance assembly as in claim 14, wherein said flat layer further defines said needle safety cap port hole to cooperate accurately with the exterior shape of the anesthetic needle assembly distal safety cap, thereby allowing the anesthetic needle assembly distal safety cap to reversibly mount onto said needle safety cap port hole, yielding a “slip fit” between said needle safety cap port hole and the anesthetic needle assembly distal safety cap.

16. A device to support said pre-angled needle guidance assembly as in claim 1 5 wherein said device to support said pre-angled needle guidance assembly is made of stainless steel or other autoclaveable material including but not limiting to metal, plastic, ceramic, fiberglass, or any combination thereof.

17. An oral cavity liquid delivery system, comprising: said pre-angled needle guidance assembly; said cartridge ampule; an anesthetic syringe; and an anesthetic needle assembly reversibly attachable to said anesthetic syringe.

18. An oral cavity liquid delivery system as in claim 17, wherein said anesthetic syringe further comprises: a body member having means providing a cavity to accommodate therewithin one of said cartridge ampules, and having axially disposed openings at the front and rear ends thereof; a plunger member extending through the opening at the rear end of said cavity and slidable with respect thereto, said plunger having a head at its forward end adapted to engage said movable piston member to slide the same forwardly and rearwardly within said tubular body, and a manually engageable actuator at the rear end thereof for pressing said plunger forward; and an adaptor within the opening at the front end of the syringe body member, said adapter being arranged to receive and removably secure an anesthetic needle assembly with axial alignment between said tubular body and said proximal portion of an anesthetic needle assembly, wherein the same extends through said flexible membrane into said certain sterile fluid.

19. An oral cavity liquid delivery system, comprising: said pre-angled needle guidance assembly; said cartridge ampule; an intraligamental syringe; and an anesthetic needle assembly reversibly attachable to said intraligamental syringe.

20. A method for performing an oral cavity injection using said pre-angled needle guidance assembly, comprising the steps of: loading an anesthetic cartridge ampule into a syringe; mounting an anesthetic needle assembly onto the syringe; removing the distal safety cap from said anesthetic needle assembly; mounting said pre-angled needle guidance assembly onto said anesthetic needle assembly; performing an oral cavity injection; removing said pre-angled needle guidance assembly from the anesthetic needle assembly; and recapping the distal needle on said anesthetic needle assembly with the distal safety cap.

21. A method for performing an oral cavity injection as defined by claim 20, wherein said step of mounting said pre-angled needle guidance assembly comprises: choosing the appropriate pre-angled needle guidance assembly from the oral cavity liquid delivery system with appropriate angle and appropriate tube length to perform the specific oral cavity injection procedure at hand; mounting said appropriate pre-angled needle guidance assembly into said needle docking station so that said ridges on said appropriate pre-angled needle guidance assembly straddle the thickness of said horizontal support member of said needle docking station and are pushed firmly against the narrow end of said keyhole mounting slot; inserting the distal needle tip of said anesthetic needle assembly several millimeters into said appropriate pre-angled needle guidance sleeve; stabilizing said needle docking station with practitioner's non-dominant hand; advancing said anesthetic needle assembly fully into said appropriate pre-angled needle guidance assembly until fully seated into said concave surface of proximal portion of said appropriate pre-angled needle guidance assembly; sliding the assembled combination of said appropriate pre-angled needle guidance assembly seated onto said anesthetic needle assembly horizontally to the wide opening in said keyhole mounting slot; and removing said assembled combination from said needle docking station.

22. A method for performing an oral cavity injection as defined by claim 20, wherein said step of performing an oral cavity injection comprises: inserting said anesthetic needle into the pulp chamber; expressing anesthetic into pulp to accomplish an intra-pulpal injection; and removing said anesthetic needle from the oral cavity.

23. A method for performing an oral cavity injection as defined by claim 20, wherein said step of performing an oral cavity injection comprises: inserting said anesthetic needle into the periodontal ligament space; pressing and otherwise supporting said anesthetic needle apically and towards the mesio-distal center of the tooth with fingers of the non-dominant hand positioned on said pre-angled needle guidance assembly; expressing anesthetic to accomplish an intraligamental injection; and removing said anesthetic needle from the oral cavity.

24. A method for performing an oral cavity injection as defined by claim 20, wherein said step of removing said pre-angled needle guidance assembly from said anesthetic needle assembly comprises: mounting said pre-angled needle guidance assembly into said needle docking station so that said ridges on said pre-angled needle guidance assembly straddle the thickness of said horizontal support member of said needle docking station and are pushed firmly against the narrow end of said keyhole mounting slot, and pulling the syringe upwardly out of said pre-angled needle guidance assembly with the dominant hand while stabilizing said needle docking station with non-dominant hand until clear of pre-angled needle guidance assembly.

25. A method for performing an oral cavity injection as defined by claim 20, wherein said step of recapping the distal needle on said anesthetic needle assembly with the distal safety cap comprises: seating the distal safety cap into said needle safety cap port hole; and recapping said anesthetic needle with the distal safety cap using a single-handed technique.

26. A method for performing an oral cavity irrigation using said pre-angled needle guidance assembly, comprising the steps of: loading a plunger-type tubular cartridge ampule into the syringe; mounting an anesthetic needle assembly onto the syringe; removing the distal safety cap from said anesthetic needle assembly; mounting said pre-angled needle guidance assembly onto said anesthetic needle assembly; performing an oral cavity irrigation; removing said pre-angled needle guidance assembly from said anesthetic needle assembly; and recapping the distal needle on said anesthetic needle assembly with the distal safety cap.

27. A method for performing an oral cavity irrigation as defined by claim 26, wherein said step of loading a plunger-type tubular cartridge ampule into the syringe comprises: choosing the appropriate plunger-type cartridge ampule from the oral cavity liquid delivery system with appropriate certain sterile fluid to perform the specific oral cavity irrigation procedure at hand; and loading said appropriate plunger-type cartridge ampule into a conventional syringe.

28. A method for performing an oral cavity irrigation as defined by claim 26, wherein said step of mounting said pre-angled needle guidance assembly comprises: choosing the appropriate pre-angled needle guidance assembly from the oral cavity liquid delivery system with appropriate angle and appropriate tube length to perform the specific oral cavity irrigation procedure at hand; mounting said appropriate pre-angled needle guidance assembly into said needle docking station so that said ridges on said appropriate pre-angled needle guidance assembly straddle the thickness of said horizontal support member of said needle docking station and are pushed firmly against the narrow end of said keyhole mounting slot; inserting the distal needle tip of said anesthetic needle several millimeters into said appropriate pre-angled needle guidance sleeve; stabilizing said needle docking station with practitioner's non-dominant hand; advancing said anesthetic needle assembly fully into said appropriate pre-angled needle guidance assembly until fully seated into said concave surface of proximal portion of said appropriate pre-angled needle guidance assembly; sliding the assembled combination of said appropriate pre-angled needle guidance assembly seated onto said anesthetic needle assembly horizontally to the wide opening in said keyhole mounting slot; and removing said assembled combination from said needle docking station.

29. A method for performing an oral cavity irrigation as defined by claim 26, wherein said step of performing an oral cavity irrigation comprises: directing the distal tip of said pre-angled needle guidance assembly towards the handpiece bur during tooth sectioning or bone removal procedures or into the extraction site after tooth removal has been accomplished; depressing the plunger of the syringe to force liquid out of the distal tip of said pre-angled needle guidance assembly into the operating site; and removing said pre-angled needle guidance assembly from the oral cavity.

30. A method for performing an oral cavity irrigation as defined by claim 26, wherein said step of performing an oral cavity irrigation comprises: directing the distal tip of said pre-angled needle guidance assembly into the pulp chamber or the coronal portion of a root canal preparation area; depressing the plunger of the syringe to force liquid out of said distal tip of pre-angled needle guidance assembly into the root canal preparation area; and removing said pre-angled needle guidance assembly from the oral cavity.

31. A method for performing an oral cavity irrigation as defined by claim 26, wherein said step of removing said pre-angled needle guidance assembly from said anesthetic needle assembly comprises: mounting said pre-angled needle guidance assembly into said needle docking station so that said ridges on said pre-angled needle guidance assembly straddle the thickness of said horizontal support member of said needle docking station and are pushed firmly against the narrow end of said keyhole mounting slot, and pulling the syringe upwardly out of said pre-angled needle guidance assembly with the dominant hand while stabilizing said needle docking station with non-dominant hand until clear of pre-angled needle guidance assembly.

32. A method for performing an oral cavity irrigation as defined by claim 26, wherein said step of recapping said distal needle on the anesthetic needle assembly with the distal safety cap comprises: seating the distal safety cap into said needle safety cap port hole; and recapping said anesthetic needle with the distal safety cap using a single-handed technique.
